(19) United States
(12) Patent Application Publication
(10) Pub. No.: US 2025/0312177 A1
(43) Pub. Date: Oct. 9, 2025

(54) MOUNTING ASSEMBLY FOR CRIMPING A PROSTHETIC VALVE

(71) Applicant: EDWARDS LIFESCIENCES CORPORATION, Irvine, CA (US)

(72) Inventor: Michael C. Murad, Lake Mathews, CA (US)

(73) Assignee: EDWARDS LIFESCIENCES CORPORATION, Irvine, CA (US)

(21) Appl. No.: 19/233,173

(22) Filed: Jun. 10, 2025

Related U.S. Application Data
(63) Continuation of application No. PCT/US2023/082775, filed on Dec. 6, 2023.
(60) Provisional application No. 63/476,296, filed on Dec. 20, 2022.

(51) Int. Cl. A61F 2/95

(52) U.S. Cl. CPC A61F 2/9524

(57) ABSTRACT

A mounting assembly for crimping a prosthetic valve onto a valve mounting portion of a delivery apparatus is disclosed. The mounting assembly includes a coupling member configured to releasably couple to a crimping device, the coupling member comprising a lumen, and a valve support member configured to be inserted into the prosthetic valve so that leaflets of the prosthetic valve contact and rest upon a support surface of the valve support member. The valve support member can be axially movable relative to the coupling member through the lumen of the coupling member.

### CROSS-REFERENCE TO RELATED APPLICATIONS

This application is a continuation of PCT Patent Application No. PCT/US2023/082775 filed on Dec. 6, 2023, which claims the benefit of U.S. Provisional Application No. 63/476,296, filed Dec. 20, 2022, the entirety of each of these applications being incorporated herein by this specific reference.

### FIELD

The present disclosure concerns apparatuses, systems, and methods for crimping a prosthetic valve on a delivery apparatus.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve (or simply “prosthetic valve”) can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size. Mounting and crimping a prosthetic heart valve on a delivery apparatus involves complex steps and requires specialized skills. Accordingly, improvements to systems and methods to facilitate such mounting and crimping operations are desirable.

### SUMMARY

Described herein are systems and methods for mounting and crimping a prosthetic heart valve on a delivery apparatus, which is configured to deliver the prosthetic heart valve, through the body and into the heart for implantation therein. The prosthetic heart valves delivered with the delivery systems disclosed herein are, for example, radially expandable from a radially compressed state mounted on the delivery system to a radially expanded state for implantation using an inflatable balloon (or an equivalent expansion device) of the delivery system. Exemplary delivery routes through the body and into the heart include transfemoral routes, transapical routes, and transaortic routes, among others. Although the devices and methods disclosed herein are particular suited for implanting prosthetic heart valves (for example, a prosthetic aortic valve or prosthetic mitral valve), the disclosed devices and methods can be adapted for implanting other types of prosthetic valves within the body (for example, prosthetic venous valves) or other types of expandable prosthetic devices adapted to be implanted in various body lumens.

In one aspect, a mounting assembly for crimping a prosthetic valve onto a valve mounting portion of a delivery apparatus is disclosed. The mounting assembly can include a coupling member configured to releasably couple to a crimping device. In addition, a mounting assembly can further comprise one or more of the components disclosed herein.

In some examples, a mounting assembly can include a valve support member configured to be inserted into the prosthetic valve so that leaflets of the prosthetic valve contact and rest upon a support surface of the valve support member. In some examples, the coupling member can comprise a lumen. The valve support member can be axially movable relative to the coupling member through the lumen of the coupling member.

In some examples, a mounting assembly can include a valve support member comprising a connect portion inserted into the coupling member and a support surface. The prosthetic valve can slide over the support surface so that leaflets of the prosthetic valve contact and rest upon the support surface. The valve support member can be axially movable between an extended state and a retracted state. The support surface can extend out of the coupling member when the valve support member is in the extended state. The support surface can be at least partially covered by the coupling member when the valve support member is in the retracted state.

In some examples, a mounting assembly can include a valve support member comprising a connect portion connected to the coupling member and a support surface. The valve support member can be movable between an extended state and a retracted state. When the valve support member is in the extended state, the prosthetic valve in a radially expanded configuration can slide over the support surface. When the valve support member is in the retracted state, the support surface is moved away from the prosthetic valve so that the prosthetic valve can be crimped to a radially compressed configuration by the crimping device.

In another aspect, a method of mounting a prosthetic valve onto a valve mounting portion of a delivery apparatus is disclosed. The method can include placing the prosthetic valve in a radially expanded configuration over a support surface of a valve support member. A connect portion of the valve support member can be inserted into a coupling member.

In some examples, the method can include inserting the prosthetic vale placed over the support surface into a channel of a crimping device, the channel being surrounded by a plurality of pressing surfaces of the crimping device. The method can further include attaching the coupling member to the crimping device, and radially compressing the prosthetic valve placed within the channel by actuating the crimping device to move the pressing surfaces radially inwardly. The radially compressing causes the support surface to be ejected out of the prosthetic valve while the coupling member remains fixed relative to the crimping device.

The above method(s) can be performed to prepare a delivery apparatus for implanting a prosthetic valve in a living animal or on a simulation, such as on a cadaver, cadaver heart, anthropomorphic ghost, simulator (for example, with body parts, heart, tissue, etc. being simulated).

In some examples, a mounting assembly comprises one or more of the components recited in Examples 1-25 described in the section “Additional Examples of the Disclosed Technology” below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side perspective view of a prosthetic heart valve, according to one example.

FIG. 2A is a side view of an example delivery apparatus configured to deliver and implant a radially expandable prosthetic heart valve at an implantation site.

FIG. 2B is a cross-sectional side view of a distal end portion of the delivery apparatus of FIG. 2A.

FIG. 3A is a rear perspective view of an example crimping device configured to crimp a prosthetic valve onto a portion of a delivery apparatus.

FIG. 3B is a front perspective view of the crimping device of FIG. 3A.

FIG. 4 is a perspective view of a mounting assembly comprising a coupling member and a valve support member, as well as a ring body, according to one example.

FIG. 5 is another perspective view of the mounting assembly of FIG. 4, without showing the ring body.

FIG. 6 is a perspective view of the coupling member of the mounting assembly of FIG. 4.

FIG. 7 is a perspective view of the valve support member of the mounting assembly of FIG. 4.

FIG. 8 is a side cross-sectional view of the mounting assembly of FIG. 5, where the valve support member is in an extended state.

FIG. 9 is a side cross-sectional view of the mounting assembly of FIG. 5, where the valve support member is in a retracted state.

FIG. 10A is a front perspective view of the ring body depicted in FIG. 4, according to one example.

FIG. 10B is a rear perspective view of the ring body of FIG. 10A.

FIG. 11 is a perspective view of a positioning device coupled to a distal end portion of a delivery apparatus, according to one example.

FIG. 12 is an end view of a prosthetic valve mounted on the valve support member of the mounting assembly of FIG. 4, with commissure aligned with corresponding indicators on the ring body.

FIG. 13 is a cross-sectional view of the mounting assembly of FIG. 4, coupled to and arranged within the crimping device of FIG. 3A such that the prosthetic valve is arranged in a predetermined orientation and/or position around a valve mounting portion of the delivery apparatus.

FIG. 14 is a cross-sectional view of the prosthetic valve radially compressed onto the valve mounting portion of the delivery apparatus, after performing a crimping operation with the crimping device of FIG. 3A.

### DETAILED DESCRIPTION

### General Considerations

It should be understood that the disclosed examples can be adapted to deliver and implant prosthetic devices in any of the native annuluses of the heart (for example, the pulmonary, mitral, and tricuspid annuluses), and can be used with any of various delivery approaches (for example, retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (for example, mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As described herein, the term “inflow” can generally refer to a position, direction, or portion of the prosthetic heart valve that is closer to an inlet into which blood flow enters the prosthetic heart valve. As described herein, the term “outflow” can generally refer to a position, direction, or portion of a prosthetic heart valve that is closer to an outlet from which blood flow exits the prosthetic heart valve.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Overview of an Exemplary Prosthetic Valve

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

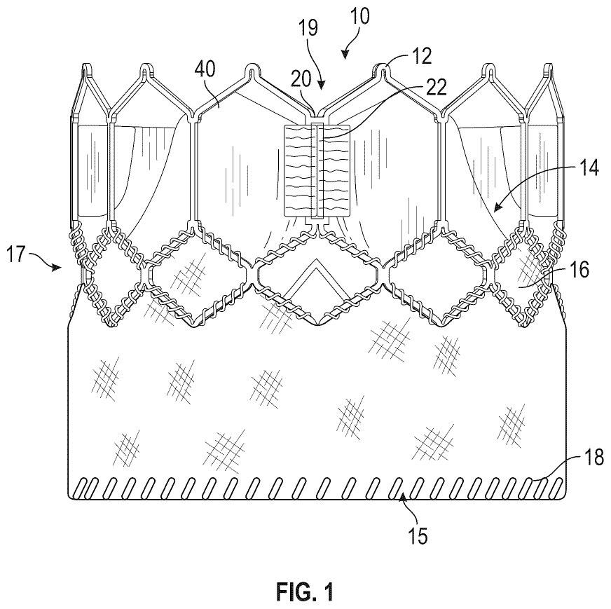

FIG. 1 shows a prosthetic valve 10, according to one example. The prosthetic valve 10 can include a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the valvular (for example, leaflet) structure 14. The lower edge of valvular structure 14 can have an undulating, curved scalloped shape, and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame.

The frame 12 can be made of any of various suitable plastically-expandable materials (for example, stainless steel, etc.) or self-expanding materials (for example, Nitinol) as known in the art. When constructed of a plastically-expandable material, the frame 12 (and thus the valve 10) can be crimped to a radially compressed state on a delivery catheter and then expanded inside a patient by an inflatable balloon or equivalent expansion mechanism. When constructed of a self-expandable material, the frame 12 (and thus the valve 10) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details regarding the prosthetic valve 10 and its various components are described in WIPO Patent Application Publication No. WO 2018/222799, which is incorporated herein by reference in its entirety. Additional variance of the prosthetic valve and different valve components are further described in U.S. Pat. No. 9,155,619 and U.S. Patent Publication No. 2018/0028310, both of which are incorporated herein by reference in their entireties.

### Overview of an Exemplary Delivery Apparatus for Implanting a Prosthetic Valve

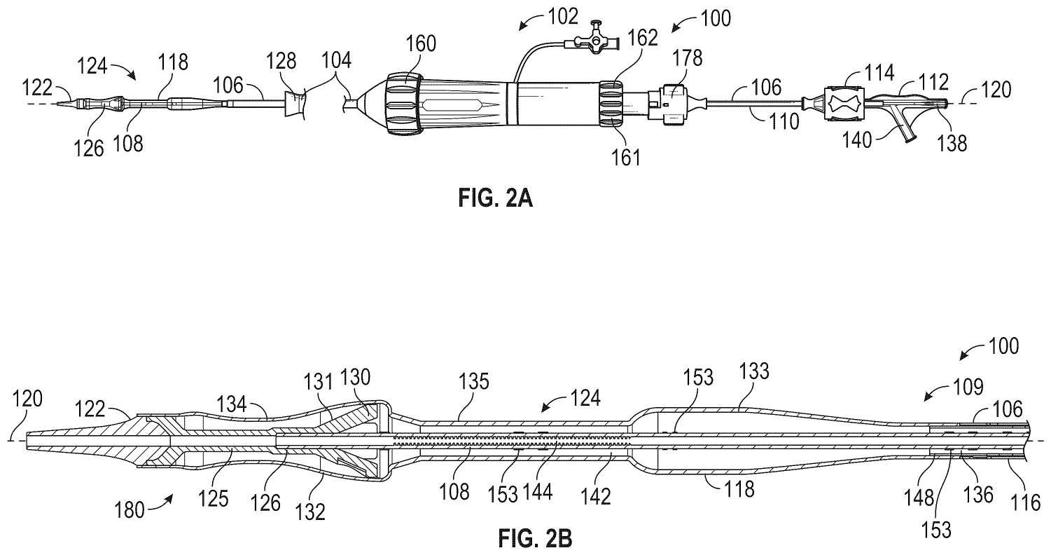

FIG. 2A shows an example delivery apparatus 100, which can be used to implant an expandable prosthetic heart valve (for example, the prosthetic valve 10 of FIG. 1), or another type of expandable prosthetic medical device (such as a stent). A distal end portion 109 of the delivery apparatus 100 is shown in FIG. 2B. In some examples, the delivery apparatus 100 is specifically adapted for use in introducing a prosthetic valve into a heart. As described further below, the delivery apparatus 100 can be configured to rotate the prosthetic valve, mounted on the delivery apparatus in a radially compressed state, at the target implantation site (for example, at a native valve of the heart) to achieve commissure alignment between the native valve and prosthetic valve after deploying the prosthetic valve.

In the depicted example, the delivery apparatus 100 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends both proximally and distally from the handle 102. The portion of the intermediate shaft 106 extending distally from the handle 102 also extends coaxially through the outer shaft 104. Additionally, the delivery apparatus 100 can further comprise an inner shaft 108 extending distally from the handle 102 and coaxially through the intermediate shaft 106 and the outer shaft 104. The inner shaft 108 also extends proximally from the handle 102 and coaxially through the intermediate shaft 106.

The outer shaft 104 and the intermediate shaft 106 are configured to translate (for example, move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110. The knob 114 can be configured to rotate the intermediate shaft 106 around the central longitudinal axis 120 of the delivery apparatus 100 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion 116 that extends distally beyond a distal end of the outer shaft 104 when the distal end of the outer shaft 104 is positioned away from an inflatable balloon 118 of the delivery apparatus. A distal end portion of the inner shaft 108 can extend distally beyond the distal end portion 116 of the intermediate shaft 106.

The balloon 118 can be coupled to the distal end portion 116 of the intermediate shaft 106. For example, a proximal end portion of the balloon 118 can be coupled to and/or around a distal end 348 of the intermediate shaft 106.

The balloon 118 can comprise a distal end portion (or section) 132, a proximal end portion (or section) 133, and an intermediate portion (or section) 135, the intermediate portion 135 disposed between the distal end portion 132 and the proximal end portion 133.

In some examples, a distal end of the distal end portion 132 of the balloon 118 can be coupled to a distal end of the delivery apparatus 100, such as to a nose cone 122, or to an alternate component at the distal end of the delivery apparatus 100 (for example, a distal shoulder). In some examples, the intermediate portion 135 of the balloon 118 can overlay a valve mounting portion 124 of a distal end portion 109 of the delivery apparatus 100, the distal end portion 132 can overly a distal shoulder 126 of the delivery apparatus 100, and the proximal end portion 133 can surround a portion of the inner shaft 108 (FIG. 2B). The valve mounting portion 124 and the intermediate portion 135 of the balloon 118 can be configured to receive a prosthetic valve in a radially compressed state.

As described herein, rotation of the intermediate shaft 106 can cause rotation of the balloon 118 and the prosthetic valve mounted thereon for rotational positioning of the prosthetic valve relative to the native anatomy at the target implantation site.

The delivery apparatus 100 can include a balloon shoulder assembly 180 configured to maintain the prosthetic heart valve or other medical device at a fixed position on the balloon 118 during delivery through the patient's vasculature. The balloon shoulder assembly 180 can include a distal shoulder 126 arranged within a distal end portion of the balloon 118 and coupled to the distal end portion of the inner shaft 108. The distal shoulder 126 can be configured to resist movement of the prosthetic valve or other medical device mounted on the valve mounting portion 124 distally, in an axial direction (for example, along the central longitudinal axis 120), relative to the balloon 118.

For example, the distal shoulder 126 can include a flared portion 131 arranged adjacent to the valve mounting portion 124. In some examples, the flared portion 131 can include a plurality of wings 130 that flare radially outward from a base portion 125 (for example, shaft) of the distal shoulder 126, toward the valve mounting portion 124.

The outer shaft 104 can include a distal tip portion 128 mounted on its distal end. In some examples, the distal tip portion 128 can be configured as a flex adaptor including a plurality of inner and outer helical grooves. The outer shaft 104 and the intermediate shaft 106 can be translated axially relative to one another to position the distal tip portion 128 adjacent to a proximal end of the valve mounting portion 124, when a prosthetic valve is mounted in the radially compressed state on the valve mounting portion 124 and during delivery of the prosthetic valve to the target implantation site. As such, the distal tip portion 128 can be configured to resist movement of the prosthetic valve relative to the balloon 118 proximally, in the axial direction, relative to the balloon 118, when the distal tip portion 128 is arranged adjacent to a proximal side of the valve mounting portion 124.

In some examples, the nose cone 122 can be disposed distal to and be coupled to the distal shoulder 126. In some examples, the nose cone 122 can be coupled to the distal end portion of the inner shaft 108.

In some examples, the delivery apparatus 100 can comprise one or more markers or marker bands 153 that are configured to indicate to a user a location of a specified component of the delivery apparatus. In some examples, the one or more marker bands 153 can be radiopaque. In some examples, one or more marker bands 153 can be radially compressed (for example, crimped) onto the inner shaft 108.

In some examples, the distal end portion 132 of the balloon 118 can include a radial depression 134 that is depressed radially inwardly, toward the central longitudinal axis 120, relative to an outermost radial surface of the distal shoulder 126 and an outermost radial surface of the nose cone 122.

An annular space 136 can be defined between an outer surface of the inner shaft 108 and an inner surface of the intermediate shaft 106. In some examples, the annular space 136 can be referred to as an inner lumen of the intermediate shaft 106. In some examples, the annular space 136 can be configured to receive an inflation fluid from a fluid source via the second port 140 of the adaptor 112 (for example, the annular space 136 can be in fluid communication with the second port 140 of the adaptor 112). The annular space 136 can be fluidly coupled to a fluid passageway 142 formed between the outer surface of the distal end portion of the inner shaft 108 and an inner surface of the balloon 118. As such, fluid from the fluid source can flow to the fluid passageway 142 from the annular space 136 to inflate the balloon 118 and radially expand and deploy the prosthetic valve.

In some examples, after crimping of the prosthetic valve onto the valve mounting portion 124, the distal tip portion 128 can be advanced over the proximal end portion 133 of the balloon 118. As a result, fluid arranged within the proximal end portion 133 of the balloon 118 can be displaced and pushed distally, within the balloon 118, to the distal end portion 132 of the balloon 118. The radially depressed, distal end portion 132 of the balloon 118 can then radially expand (for example, inflate partially) as it receives the displaced fluid to an expanded state. The radial depression 134 can be configured (for example, sized) so that the distal end portion 132 can receive the displaced fluid without radial expanding the portion of the balloon 118 within the valve mounting portion 124, thereby preventing the crimped profile of the prosthetic valve from increasing.

An inner lumen 144 of the inner shaft 108 can be configured to receive a guidewire therethrough, for navigating the distal end portion 109 of the delivery apparatus 100 to the target implantation site. As introduced above, the first port 138 of the adaptor 112 can be coupled to the inner lumen 144 and configured to receive the guidewire. For example, the distal end portion 109 of the delivery apparatus 100 can be advanced over the guidewire, to the target implantation site.

In some examples, the intermediate (for example, balloon) shaft 106 can include two layers of a braided (or coil) material that are configured to increase the torque resistance of the intermediate shaft 106 so that it can withstand rotation at the target implantation site. The braided or coil material can comprise a more rigid braided or coiled material, such as metal or polyethylene terephthalate (PET).

In some examples, the intermediate shaft 106 can have a proximal portion and a distal portion, and the proximal portion can be longer than the distal portion. For example, the length of the proximal portion can be a majority of a total length of the intermediate shaft 106. In some examples, the length of the distal portion can be in a range of 4 to 10 inches, 4 to 8 inches, or 5 to 7 inches (for example, approximately 6 inches). The two layers of the braided material of the intermediate shaft 106 can include a first braided layer that extends along an entire length of the intermediate shaft 106 (up until the distal end 148), along both the proximal portion and the distal portion. The two layers of the braided material of the intermediate shaft 106 can further include a second braided layer that extends a majority of the entire length of the intermediate shaft 106, along the proximal portion but stops before the distal portion. This can allow the distal portion of the intermediate shaft 106 to have increased flexibility at the distal end portion 116 compared to the proximal portion. In alternate examples, the second braided layer can extend the entire length of the intermediate shaft 106. In some alternate examples, the intermediate shaft 106 can include more than two layers of braided material, such as three.

As shown in FIG. 2A, the handle 102 can include a steering mechanism configured to adjust the curvature of the distal end portion 109 of the delivery apparatus 100. For example, the handle 102 can include an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion 109 of the delivery apparatus 100. Further details on steering or flex mechanisms for the delivery apparatus are described in U.S. Pat. No. 9,339,384, which is incorporated by reference herein in its entirety.

The handle 102 can further include an adjustment mechanism 161 including an adjustment member, such as the illustrated rotatable knob 162. The adjustment mechanism 161 can be configured to move (thus adjust the axial position) of the intermediate shaft 106 relative to the outer shaft 104. The handle 102 can also include a locking mechanism configured to retain (for example, lock) the position of the intermediate shaft 106 relative to the handle 102. In some examples, the locking mechanism can include another adjustment member, which can be configured as a rotatable knob 178. In some examples, rotating the knob 178 to a locked position can cause the intermediate shaft 106 to frictionally engage with other components of the handle 102, thereby restraining movement of the intermediate shaft 106 for fine positioning of the prosthetic valve mounted on the distal end portion of the delivery apparatus 100. Rotating the knob 178 to an unlocked position allows axial and rotational movement of the intermediate shaft 106 relative to the proximal end portion of the handle 102. For example, rotation of the knob 162 can cause the intermediate shaft 106 to move axially relative to the outer shaft 104 (either in the proximal or distal direction, depending on the direction the knob 162 is rotated).

Further details on the adjustment mechanism and locking mechanism of the handle 102 can be found in U.S. Pat. No. 9,339,384, which is incorporated by reference herein in its entirety. Additional examples of delivery apparatuses and related steering mechanism can be found in U.S. Pat. No. 8,568,472, which is incorporated herein by reference in its entirety.

### Example Implantation Procedure

According to certain examples, to implant a prosthetic valve (for example, prosthetic valve 10) in a native heart valve of the patient, the delivery apparatus 100 can be introduced into a vasculature of the patient. The prosthetic valve can be initially retained in a radially compressed configuration on the valve mounting portion 124 (and over the balloon 118) of the delivery apparatus 100. Once inside the patient's vasculature, the position of the prosthetic valve relative to the balloon 118 can be adjusted such that the prosthetic valve 10 is centered on the balloon 118. When navigating the prosthetic valve through an arched region of the vasculature (for example, the aortic arch), the curvature of the distal end portion 109 of the delivery apparatus 100 can be adjusted, for example, by rotating the knob 160 to increase or decrease the tension in the pull wire which extends between the handle 102 and the distal end of the outer shaft 104. The prosthetic valve can be positioned within or adjacent an annulus of the native heart valve. Prior to inflating the balloon 118, the outer shaft 104 can be retracted proximally away from the balloon 118 for a sufficient distance so that the outer shaft does not interfere with balloon inflation. This can be accomplished, for example, by holding the adaptor 112 stationary against the operating table and rotating the knob 362 in a direction that causes the handle 102 and the outer shaft 104 to move proximally away from the balloon 118. Then, the prosthetic valve can be radially expanded and deployed by inflating the balloon 118. Inflation of the balloon 118 can radially expand the prosthetic valve 10 so that the prosthetic valve 10 contacts the native annulus. The expanded prosthetic valve 10 becomes anchored within the native aortic annulus by the radial outward force of the valve's frame against the surrounding tissue.

As described above, the knob 114 of the handle 102 can be configured to rotate the intermediate shaft 106, thereby rotating the balloon 118 mounted on the intermediate shaft 106 and a radially compressed prosthetic valve mounted on the balloon 118, around the valve mounting portion 124. Thus, rotating the knob 114 can rotate the prosthetic valve, around the central longitudinal axis 120, into a desired orientation relative to the native anatomy at the target implantation site.

For example, to implant a prosthetic valve (for example, 10) at a native aortic valve, instead of deploying the prosthetic valve with the delivery apparatus 100 in a random rotational orientation relative to the aorta, which may result in commissures (for example, 22) of the prosthetic valve being arranged in front of the coronary arteries, it may be desirable to deploy the prosthetic valve in a targeted rotational orientation where the commissures are positioned away from and do not block the coronary arteries (to reduce the likelihood of reduced blood flow and/or less oxygenated blood flow into the coronary arteries and the heart muscle). Specifically, the delivery apparatus 100 can be configured to control the rotational positioning of the prosthetic heart valve relative to the native valve, to achieve the commissure alignment, thereby increasing blood flow access to the coronary arteries. Additionally, this positioning of the prosthetic heart valve can facilitate a later, leaflet cutting procedure that provides increased blood flow to the coronary arteries.

Further details methods of aligning commissures of native valve with commissures of prosthetic valve after deploying the prosthetic valve are described in PCT Patent Application No. WO 2022/046585, which is incorporated by reference herein in its entirety.

### Example Crimping Device

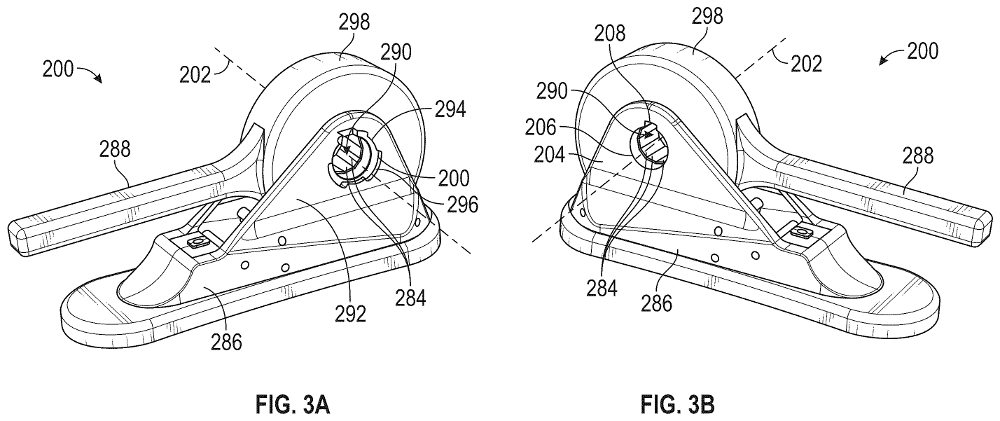

FIGS. 3-14 show examples of various components that can be used to crimp a prosthetic valve (such as prosthetic valve 10) onto a valve mounting portion of a delivery apparatus (for example, the valve mounting portion 124 of delivery apparatus 100) in a predetermined position and orientation. The prosthetic valve may be crimped to the valve mounting portion of the delivery apparatus in a variety of manners. In some examples, a crimping device, such as the crimping device 200 shown in FIGS. 3A and 3B, can be used to crimp the prosthetic valve onto the valve mounting portion of the delivery apparatus. As described further below, the crimping device 200 can include mating interfaces, on opposite sides of the crimping device 200, that are configured to receive and mate with corresponding mating interfaces on first and second components of the mounting assembly.

FIG. 3A illustrates a rear perspective view of the crimping device 200 (or a view from the proximal side of the crimping device 200) and FIG. 3B illustrates a front perspective view of the crimping device 200 (or a view from the distal side of the crimping device 200). The crimping device 200 can include a base 286, an actuator in the form of a handle 288, and a channel 290 for the prosthetic valve and the delivery apparatus to be inserted into. The crimping device 200 may include a proximal face 292 including a proximal opening 294 that leads into the channel 290. The proximal opening 294 may be configured for the delivery apparatus to be inserted into the channel 290 therethrough.

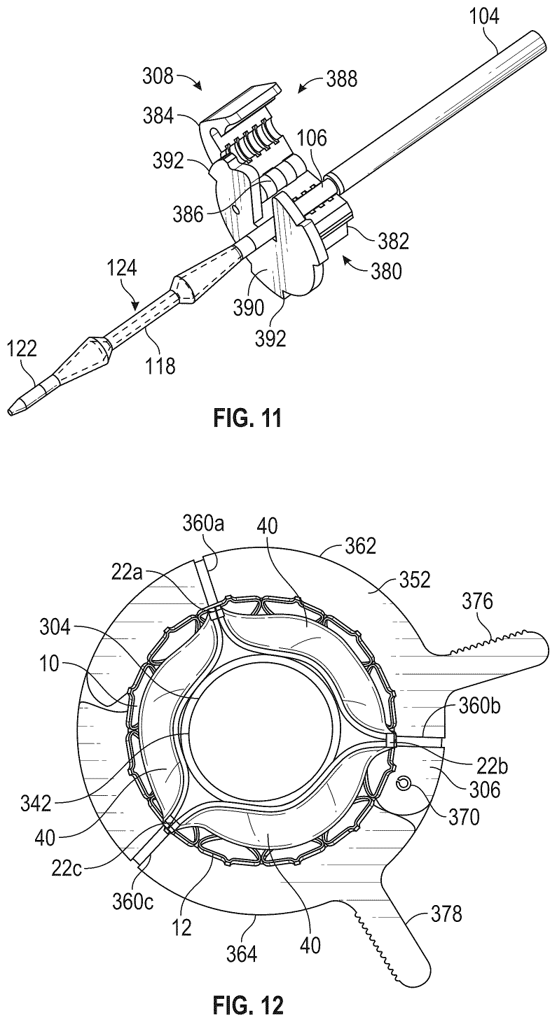

In some examples, the proximal face 292 can include a mating interface with mating structures 296 in the form of cut-outs that can be configured to mate with a positioning device (for example, 308 as shown in FIG. 11). For example, the mating interface can include one or more mating structures 296.

The crimping device 200 can further include a rotatable body 298 configured to be rotated with rotation of the handle 288. The crimping device 200 may operate by a plurality of pressing surfaces 284 surrounding the channel 290 and being configured to apply a compressive force to radially compress a prosthetic valve positioned within the channel 290 (for example, prosthetic valve 10). The pressing surfaces 284 may surround an axis 202 of the channel 290. The pressing surfaces 284 may be configured such that as the rotatable body 298 is rotated, a body presses and moves the pressing surfaces 284 towards the center of the channel 290 and the diameter of the channel 290 reduces. The pressing surfaces 284 may form an iris structure that allows the pressing surfaces 284 to move towards the center of the channel 290 and reduce the diameter of the channel 290. A prosthetic valve positioned within the channel 290 will accordingly be compressed within the channel 290, due to the radially compressive force of the pressing surfaces 284 against the prosthetic valve.

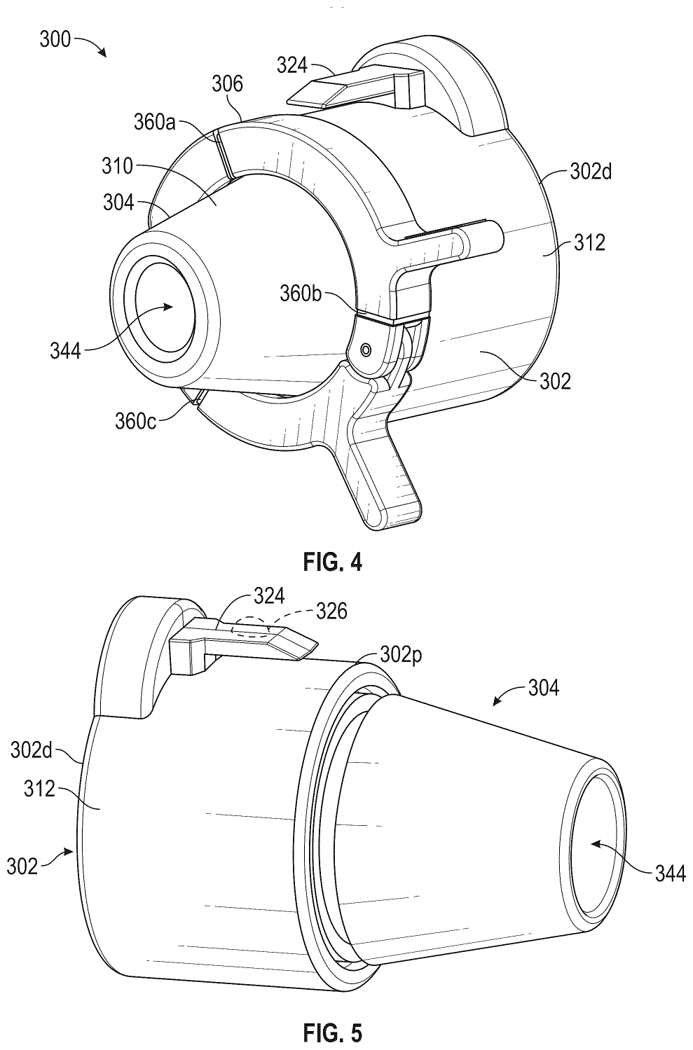

As shown in FIG. 3B, the crimping device 200 may include a distal face 204 including a distal opening 206 that leads into the channel 290. The distal face 204 can include a mating interface, which can comprise a cut-out portion 208. In some examples, the cut-out portion 208 can be configured as a notch, indentation, depression, or the like, in the distal face 204. The cut-out portion 208 can be configured (for example, shaped) to receive an alignment device of a valve support member for the prosthetic valve (for example, the projection 324 shown in FIGS. 4-6, as described further below).

The distal opening 206 can be configured for a portion of the delivery apparatus to pass therethrough during a crimping operation being performed by the crimping device 200.

The configuration of a crimping device can be varied in alternate examples.

### Example Mounting Assembly

For crimping the prosthetic valve onto the valve mounting portion of the delivery apparatus, it may be desirable to maintain the leaflets (for example, leaflets 40 of prosthetic valve 10) in an open position during crimping of the prosthetic valve to the delivery apparatus, thereby reducing a likelihood of degradation to the leaflets and/or attachments of the leaflets to a frame of the prosthetic valve. Thus, in some examples, a valve support member that is configured to support and/or maintain one or more leaflets of the prosthetic valve in an open position can be used as a component of a mounting assembly that is configured to hold the prosthetic valve and position the prosthetic valve within the crimping device.

FIG. 4 depicts a mounting assembly 300, which can be used to mount a prosthetic valve in a crimped state on a valve mounting portion of a delivery apparatus (for example, the valve mounting portion 124 of the delivery apparatus 100). In the depicted example, the mounting assembly 300 includes a coupling member 302, a valve support member 304 at least partially inserted into the coupling member 302, and a ring body 306 (also referred to herein as an alignment ring) extending around the valve support member 304.

As described more fully below, the coupling member 302 can be configured to releasably couple to a crimping device, such as the crimping device 200 shown in FIGS. 3A-3B. The valve support member 304 can be configured to be inserted into a prosthetic valve (for example, prosthetic valve 10) so that leaflets (for example, leaflets 40) of the prosthetic valve can contact and rest upon a support surface (for example, 310) of the valve support member 304. The ring body 306 can be used to space the prosthetic valve away from the coupling member 302 and achieve a desired rotational alignment of the prosthetic valve relative to the coupling member 302 and the crimping device.

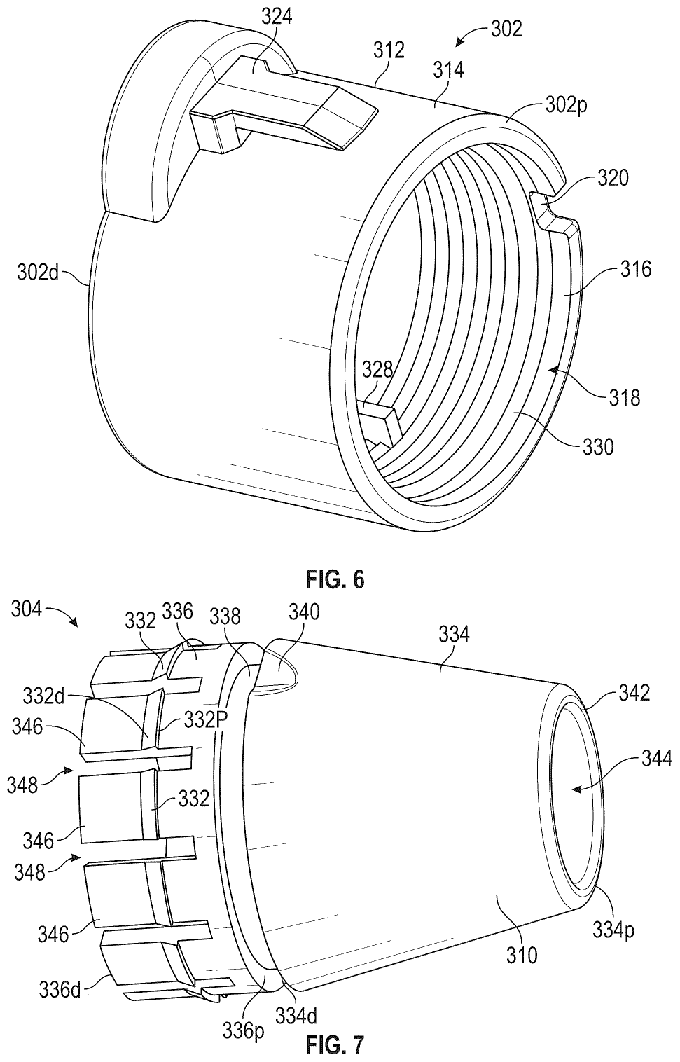

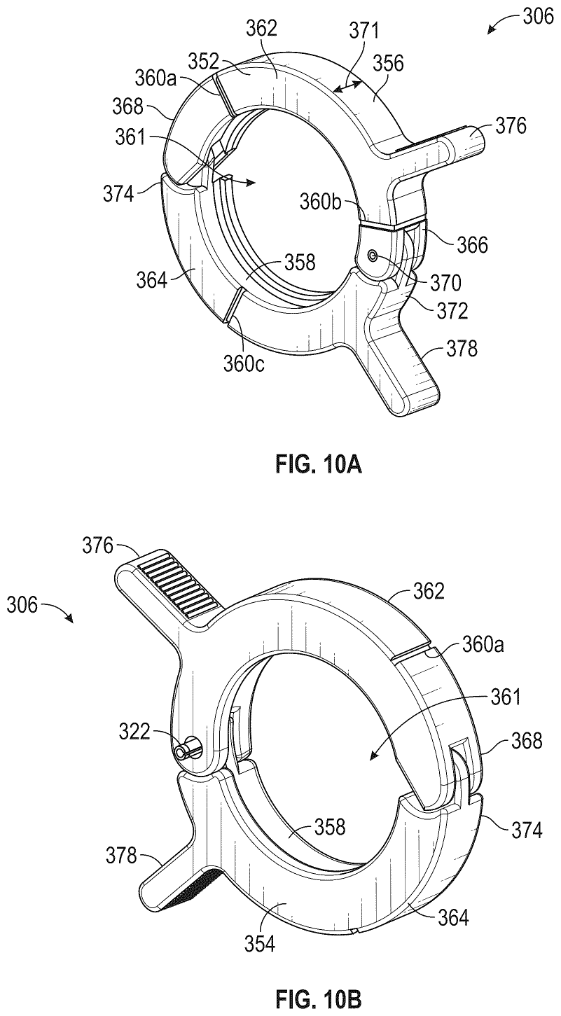

FIG. 5 is another view of the mounting assembly 300, without showing the ring body 306. FIGS. 6-7 show additional views of the coupling member 302 and the valve support member 304, respectively. FIGS. 10A-10B show additional views of the ring body 306.

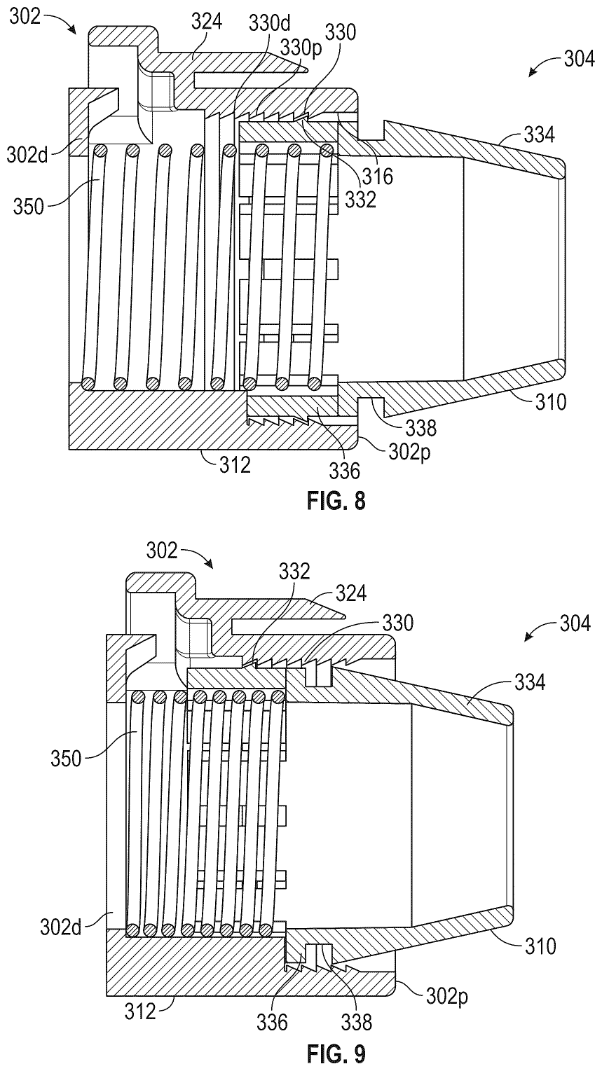

The valve support member 304 is movably coupled to the coupling member 302. As described below and illustrated in FIGS. 8-9, when the ring body 306 is removed from the valve support member 304, the valve support member 304 is configured to be axially movable relative to the coupling member 302.

In some examples, the mounting assembly 300 can further include a positioning device 308, as described further below with reference to FIG. 11.

In some examples, the mounting assembly 300 can also include a crimping device, such as the crimping device 200 depicted in FIGS. 3A-3B.

### Example Coupling Member

In some examples, the coupling member 302 can be configured to be inserted into the channel 290 of the crimping device 200.

The coupling member 302 can include a first (distal) end 302d and a second (proximal) end 302p. The valve support member 304 can include an outward-facing support surface 310 that is configured to receive the prosthetic valve thereon (for example, an interface with the valve leaflets 40).

In some examples, as shown in FIGS. 4-6, the coupling member 302 can have a generally cylindrical shape with a cylindrical wall 312 extending between the first end 302d and the second end 302p. The wall 312 has an outer surface 314 and an inner surface 316. The inner surface 316 can define a lumen 318 into which the valve support member 304 can extend.

In some examples, the wall 312 can include an alignment element, such as a cut-out 320 extending from the outer surface 314 to the inner surface 316. The cut-out 320 can be configured to receive a coupler (for example, coupling element 322) of the ring body 306, as shown in FIGS. 10A-10B.

Referring to FIG. 6, the coupling member 302 can also comprise an alignment member configured to rotationally align the coupling member 302 with the crimping device 200. The alignment member can be circumferentially positioned on the coupling member 302, proximate to the first end 302d, at a position that circumferentially aligns the coupling member 302 in a predetermined position and orientation within the channel 290 of the crimping device 200.

In some examples, as shown in FIGS. 4-6, the alignment member of the coupling member 302 comprises a projection 324 (e.g., a deflectable tab) that extends axially away from the first end 302d toward to the second end 302p of the coupling member 302 and is configured to mate with the cut-out portion 208 of the crimping device 200. In this manner, the projection 324 of the coupling member 302 rotationally aligns the coupling member 302 with the crimping device 200.

In other examples, the alignment member of the coupling member can have other configurations, such as a recess or other alignment feature that is configured to mate with a corresponding mating interface of the crimping device 200. As one example, the crimping device can comprise a projection (in lieu of the cut-out portion 208) and the coupling member can comprise a slot (in lieu of the projection 324) configured to receive the projection of the crimping device.

In some examples, the mounting assembly 300 can include an axial locking mechanism configured to prevent the coupling member 302 from moving axially relative to the crimping device 200 after the mounting assembly 300 is attached to the crimping device 200. For example, the axial locking mechanism can prevent the coupling member 302 from moving axially when the valve support member 304 moves axially relative to the coupling member 302. For example, the axial locking mechanism can be configured to prevent the projection 324 to slide distally out of the cut-out portion 208 during operation of the crimping device 200.

In one specific example, the axial locking mechanism can comprise a protrusion 326 (shown in dashed line in FIG. 5) extending radially outwardly from the projection 324 (for example, the axially extending lever) of the coupling member 302 and a recess connected to the cut-out portion 208 of the crimping device 200. When the projection 324 is inserted into the cut-out portion 208, the projection 324 and/or the protrusion 326 can elastically deform (e.g., deflect) until the protrusion 326 aligns with the recess of the crimping device. At this point, resiliency of the projection 324 and/or the protrusion 326 results in the protrusion snapping into the recess, thereby locking the coupling member 302 to the crimping device 200 and resisting axial movement therebetween. To unlock or axially separate the coupling member 302 from the crimping device 200, the coupling member 302 must be pushed or pulled away from the crimping device 200 with a sufficient force to overcome the resistance so that the protrusion 326 can escape the recess. Various other “snap-fit” connections can be used. In other examples, the axial locking mechanism can be implemented in different manners beside snap-fit (e.g., pins, fasteners etc.).

In some examples, the mounting assembly 300 can include a rotation locking mechanism configured to prevent the valve support member 304 from rotating relative to the coupling member 302.

In some examples, the rotation locking mechanism can include a key 328 extending axially along the inner surface 316 of the coupling member 302 and a channel formed on a connect portion of the valve support member 304. The channel can be configured to receive the key 328. Thus, the channel allows the valve support member 304 to move axially, but not rotate, relative to the coupling member 302. In some examples, as depicted in FIG. 7, the channel can be a gap (for example, 348) formed between two adjacent axially extending arms (for example, 346) formed on a connect portion (for example, 336) of the valve support member 304.

In some examples, the mounting assembly 300 can include a direction control mechanism configured to allow the valve support member 304 to move distally relative to the coupling member 302 (for example, moving from the second end 302p toward the first end 302d) and prevent the valve support member 304 from moving proximally relative to the coupling member 302.

In some examples, the direction control mechanism can include at least one ridge or pawl (for example, 332 of FIG. 7) extending radially outwardly from a connect portion (for example, 336) of the valve support member 304 and a plurality of teeth 330 located on the inner surface 316 of the coupling member 302. The at least one ridge is configured to matingly engage with the plurality of teeth 330.

As shown in FIG. 6, each teeth 330 can extend circumferentially around the inner surface 316. As shown in FIGS. 8-9, the plurality of teeth 330 can be arranged axially along at least in a proximal portion of the coupling member 302, where the ridges 332 of the valve support member 304 can move across.

Also as depicted in FIGS. 8-9, each teeth 330 has a distal edge 330d which has a relatively steep slope (for example, about perpendicular to the inner surface 316) and a proximal edge 330p which has a relatively more moderate slope (for example, with an acute angle relative to the inner surface 316). Thus, the ridges 332 can move in the distal direction and easily slide over the moderately sloped proximal edges 330p but are blocked by the steeply sloped distal edges 330d from moving in the proximal direction. As such, the plurality of teeth 330 and the ridges 332 can form a linear ratchet which allows unidirectional movement (in the distal direction in this example) of the valve support member 304 relative to the coupling member 302.

### Example Valve Support Member

The valve support member 304 can be configured to be positioned between one or more leaflets of the prosthetic device and the delivery apparatus (for example, delivery apparatus 100) and for supporting the one or more leaflets in an open position.

As shown in FIG. 7, the valve support member 304 can comprise a support portion 334, a connect portion 336, and a neck portion 338 connecting the support portion 334 and the connect portion 336. The support portion 334 can define the support surface 310. The valve support member 304 can be movably coupled to the coupling member 302 through the connect portion 336.

In some examples, the support portion 334 (and thus the support surface 310) can have a tapered shape that tapers radially inwardly in a direction from a first (distal) end 334d of the support portion 334 to a second (proximal) end 334p of the support portion 334. For example, a diameter of the support portion 334 can decrease from the first end 334d to the second end 334p. In some examples, the support portion 334 can have a conical shape, as shown in FIG. 7. In alternate examples, the support portion 334 can have another shape that tapers as described above, such as hexagonal, pyramidal, etc. Because of the tapered shape, a distal portion of the support surface 310 has a larger diameter than a proximal portion of the support surface 310. In some examples, the support portion 334 can have a recess 340 located at or adjacent the first (distal) end 334d. The recess 340 can be used for rotationally aligning the valve support member 304 with the coupling member 302, as described further below.

In some examples, the neck portion 338 has a cylindrical shape with a diameter smaller than the distal end 334d of the support portion 334 and a proximal end 336p of the connect portion 336. In some examples, the neck portion 338 is configured to receive the ring body 306.

As shown in FIGS. 8-9, the valve support member 304 is configured to be axially movable relative to the coupling member 302 through the lumen 318 of the coupling member 302. For example, the valve support member 304 can be axially movable between an extended state and a retracted state. When the valve support member 304 is in the extended state, as depicted in FIG. 8, the support surface 310 extends out of the coupling member 302. When the valve support member 304 is in the retracted state, as depicted in FIG. 9, the support surface 310 is at least partially covered by the wall 312 of the coupling member 302.

The support surface 310 can be configured so that interior surfaces of the leaflets 40 of the prosthetic valve 10 can contact and rest upon the support surface 310 when the prosthetic valve 10 is positioned around the valve support member 304 (as shown in FIG. 12). The support surface 310 can be configured to resist the leaflets 40 from moving to a closed position when the prosthetic valve 10 is positioned around the valve support member 304 and within the crimping device 200.

The tapered shape of the support portion 334, as described above, can allow the valve support member 304 to slide distally and away from the crimping device 200, when the pressing surfaces 284 of the crimping device 200 press upon the support surface 310. As such, the tapered shape of the support portion 334 can cause a pressing force applied by the pressing surfaces 284 to move proximally along the tapered shape of the support surface 310, thereby moving the valve support member 304 distally and out of the channel 290 of the crimping device 200. The support surface 310 can maintain the leaflets 40 in an open position as the pressing surfaces 284 press against the tapered support surface 310.

In this way, the valve support member 304 can be configured to slide axially away from the prosthetic valve 10 during and as a result of the crimping device 200 crimping the prosthetic valve 10. The valve support member 304, for example, can be configured to be inserted into the channel 290 of the crimping device 200 and slide axially away from the channel 290 upon the crimping device 200 crimping the prosthetic valve 10, and thus, may slide in an axially distal direction (as shown in FIG. 14).

As shown in FIG. 7, the valve support member 304 can include a central aperture 342 leading to a central channel 344. The central aperture 342 and central channel 344 may be configured for the delivery apparatus to extend therethrough. An inner surface of the valve support member 304 can define the central channel 344. The central aperture 342 can be positioned at the second (proximal) end 334p of the support portion 334, and the central channel 344 can extend from the proximal end 334p of the support portion 334 to a distal end 336d of the connect portion 336.

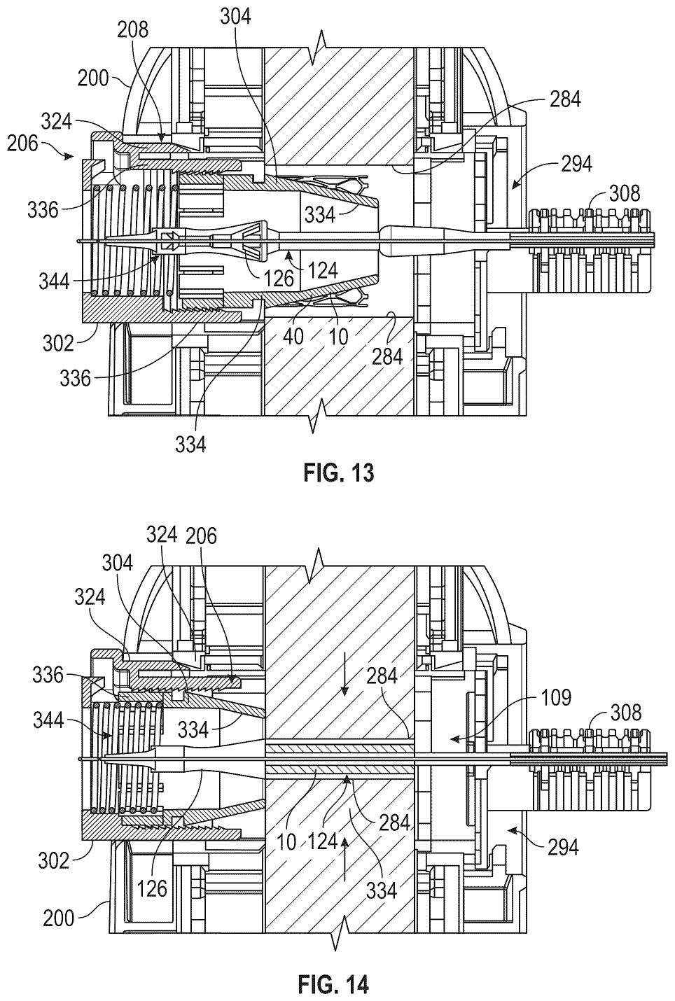

In operation, the prosthetic valve 10 may be slid distally onto the support surface 310 of the valve support member 304, with the frame 12 of the prosthetic valve 10 extending over the support surface 310 and the inner surfaces of the leaflets 40 of the prosthetic valve 10 being arranged against the support surface 310 (FIGS. 12-13).

As described herein, the valve support member 304 can remain connected to the coupling member 302 during the process of crimping the prosthetic valve 10 onto the valve mounting portion 124 of delivery apparatus 100. Coupling between the valve support member 304 and the coupling member 302 can be achieved, for example, by mating engagement between one or more ridges 332 (FIG. 7) located on the connect portion 336 and the plurality of teeth 330 (FIG. 6) located on the inner surface of the coupling member 302. The connect portion 336 of the valve support member 304 can remain inside the lumen 318 of the coupling member 302, whether the valve support member 304 is in the extended state or the retracted state. For example, when the valve support member 304 is in the extended state, the ridges 332 of the valve support member 304 can matingly engage the most proximal tooth 330 located on the inner surface 316. When the valve support member 304 is in the retracted state, the ridges 332 of the valve support member 304 can matingly engage the most distal tooth 330 located on the inner surface 316.

In some examples, as depicted in FIG. 7, the connect portion 336 can include a plurality of axially extending arms 346 arranged in a circumferential direction and separated by a plurality of axially extending gaps 348. In some examples, one of the gaps 348 can be configured as a channel which receives the key 328 of the coupling member 302, thus forming the rotation locking mechanism, as described above.

In some examples, the connect portion 336 can have one or more ridges 332 configured to matingly engage with the teeth 330 on the inner surface 316 of the coupling member. In the example depicted in FIG. 7, each axially extending arm 346 comprises a corresponding ridge 332 extending radially outwardly from the arm 346. In other examples, only some of the arms 346 (for example, every other arm, etc.) have corresponding ridges 332.

In some examples, each ridge 332 can have a proximal edge 332p which has a steep slope (for example, about perpendicular to the corresponding arm 332) and a distal edge 332d which has a more moderate slope (for example, with an acute angle relative to the corresponding arm 332). Thus, when moving the valve support member 304 distally relative to the coupling member 302, the moderately sloped distal edges 332d can facilitate sliding of the ridges 332 over the moderately sloped proximal edges of the teeth 330. Once the ridges 332 are matingly engaged with (or received in) one of the teeth 330, the steeply sloped proximal edge 332p of the ridges 332 can abut the steeply sloped distal edge 330d of the corresponding tooth 330, thus preventing the valve support member 304 from moving proximally relative to the coupling member 302.

In some examples, the axially extending arms 346 can be radially deflectable to facilitate axial movement of the valve support member 304 relative to the coupling member 302. For example, when the ridges 332 are matingly engaged with (or received in) one of the teeth 330, the arms 346 at the connect portion 336 can define a first diameter. The arms 346 can be deflected radially inwardly so as to define a second diameter (which is smaller than the first diameter) when the ridges 332 move across two adjacent teeth 330, for example, when sliding over a sloped proximal edge 330p of a first tooth to engage with a second tooth that is distal and adjacent to the first tooth.

In some examples, the mounting assembly 300 can optionally include a bias member 350, such as a coiled spring, which can be inserted into the connect portion 336. The bias member 350 can be configured to press the connect portion 336 radially outwardly against the inner surface 316 of the coupling member 302 so as to prevent the ridges 332 from rattling when the ridges 332 matingly engage (or received in) any of the teeth 330. In some examples, a distal part of the bias member 350 can extend out of the connect portion 336 and into the lumen 318 of the coupling member 302. A distal end of the bias member 350 can be anchored at or fixedly attached to the distal end 302d of the coupling member 302.

### Example Ring Body

To align the prosthetic valve 10 in a desired circumferential orientation around the valve support member 304, and to space the prosthetic valve 10 from the second (proximal) end 302p of the coupling member 302 at a desired spacing, a ring body (which can also be referred to as an alignment ring) can be utilized and positioned upon the valve support member 304.

FIGS. 10A-10B, for example, illustrate perspective views, from different sides, of a ring body 306 that can be utilized with the valve support member 304. The ring body 306 can be configured to couple to and extend around the valve support member 304. The ring body 306 can include a first surface (which may be a proximally facing surface) 352, a second surface 354 facing opposite the first surface 352 (which may be a distally facing surface), and an outer (for example, circumferential) surface 356 facing radially outward and connecting the first surface 352 to the second surface 354. The ring body 306 can include an inner surface 358 facing opposite the outer surface 356 and facing radially inward, the inner surface 358 defining a central channel (for example, opening or aperture) 361 of the ring body 306.

In some examples, an alignment guide can be positioned on the ring body 306. The alignment guide can comprise one or more indicators 360a-c (which may also be referred to as alignment markers) configured to indicate a desired circumferential (for example, rotational) position of selected elements (for example, commissures 22) of the prosthetic valve 10 relative to the ring body 306. Each indicator 360a-c may further indicate the desired circumferential position of the selected elements (for example, commissures 22) of the prosthetic valve 10 relative to the coupling member 302, as described further below.

Each indicator 360a-c can comprise a marking, groove, raised element, or other form of indicator, on one or more of the first surface 352, the second surface 354, or the outer surface 356 of the ring body 306. One or more or each of the indicators 360a-c, for example, can comprise a variation in the surface profile of the ring body 306, such as a raised portion or a recessed portion (for example, groove). For example, each of the indicators 360a-c can comprise recessed portions in the form of grooves on the first surface 352 and extending to the outer surface 356. In some examples, the indicators 360a-c can additionally be printed upon to vary a color of the respective indicator 360a-c such that the indicator is easier to visualize. In some examples, the indicators 360a-c may solely be printed upon the ring body 306 without using a variation of the surface profile (for example, without grooves).

The indicators 360a-c can be circumferentially spaced apart from each other on the ring body 306. In some examples, the indicators 360a-c can be equally spaced apart from each other around the circumference of the ring body 306. The circumferential position of each indicator 360a-c can correspond to and indicate a desired position of one of the commissures 22 of the prosthetic valve 10 when the ring body 306 is coupled to the valve support member 304 and the prosthetic valve 10 is arranged around the valve support member 304 (for example, as shown in FIG. 12). As such, a user may position the ring body 306 on the valve support member 304 and align the commissures 22a-c of the prosthetic valve 10 with respective indicators 360a-c (FIG. 12).

In some examples, the ring body 306 can include one or more arms (which can also be referred to as body portions) 362, 364 each extending around and defining the central channel 361. Each arm 362, 364 can have an arcuate shape forming the ring body 306. Each arm 362, 364 can comprise half of the ring body 306 or another amount as desired.

The first arm 362 can include a first end portion 366 and a second end portion 368, with the first end portion 366 positioned at a pivot 370 that couples the first arm 362 to the second arm 364. The second end portion 368 of the first arm 362 may include a coupler for coupling to the second arm 364. The second arm 364 may include a first end portion 372 positioned at the pivot 370 and a second end portion 374 positioned at the coupler. The coupler (also can be referred to as a coupling interface) may comprise a recess in the second end portion 368 of the first arm 362, and a protrusion at the second end portion 374 of the second arm 364. The protrusion may extend into the recess and may be held in position with an interference fit or another form of coupling. As such, the second end portions 368, 374 of the respective first arm 362 and second arm 364 may be configured to couple to each other to hold the ring body 306 together. If desired, the ring body 306 may be separated and removed from the valve support member 304 by the second end portions 368, 374 being separated from each other and the arms 362, 364 pivoted about the pivot 370 to an open position. For example, the ring body 306 can be opened to be removed from the valve support member 304 and can be closed to be held around and coupled to the valve support member 304.

As shown in FIGS. 10A-10B, a first lever (for example, radial extension) 376 may extend radially outward from the first arm 362, and a second lever (for example, radial extension) 378 may extend radially outward from the second arm 364. The first lever 376 and second lever 378 may each be configured to be pressed to rotate the first arm 362 or the second arm 364 about the pivot 370 to cause the ring body 306 to move to the open position.

In some examples, when inserting the valve support member 304 into the lumen 318 of the coupling member 302, the valve support member 304 can be rotated so that the recess 340 on the support portion 334 is aligned with the cut-out 320 on the coupling member 302. Such rotationally alignment can be configured to ensure that the key 328 in the coupling member 302 is received in the channel (for example, one of the gaps 348) of the valve support member 304 so that the valve support member 304 can move axially, but cannot rotate, relative to the coupling member 302, as described above.

In some examples, the ring body 306 can include a coupler 322 extending axially outward from the second surface 354. In some examples, the coupler 322 can be a protrusion configured to extend into the cut-out 320 of the coupling member 302 (FIG. 6). In alternate examples, the coupler 322 can be a differently shaped mating feature configured to mate with a corresponding feature on the coupling member 302.

The coupler 322 may be circumferentially positioned relative to the cut-out 320 such that the ring body 306 mates with the coupling member 302 at a desired circumferential alignment. In this way, the coupler 322 and the cut-out 320 may rotationally align the ring body 306 with the coupling member 302 so that the prosthetic valve is circumferentially aligned in a desired orientation relative to the coupling member 302 and the crimping device.

In operation, the ring body 306 can be positioned upon and/or around the valve support member 304, with the indicators 360a-c positioned at a desired rotational (for example, circumferential) alignment relative to the coupling member 302 (FIG. 4). The coupler 322 can be received within the cut-out 320, thereby circumferentially aligning the ring body 306 at a desired position relative to the coupling member 302. In other examples, other alignment devices may be utilized to rotationally align the ring body 306 with respect to the coupling member 302 in the desired rotational alignment.

In some examples, the coupler 322 can also extend axially outward from the first surface 352. The recess 340 on the support portion 334 of the valve support member 304 can be configured to receive the coupler 322 protruding out of the first surface 352. Thus, when the cut-out 320 on the coupling member 302 is rotationally aligned with the recess 340 on the support portion 334, the axially extending coupler 322 can be received within both the cut-out 320 and the recess 340.

The ring body 306 can abut the second (proximal) end 302p of the coupling member 302. The ring body 306 can be configured to abut the prosthetic valve 10 when the prosthetic valve 10 is positioned on the valve support member 304. As such, the prosthetic valve 10 may be positioned on the support surface 310 with an end of the prosthetic valve 10 abutting the first surface 352 of the ring body 306 and defining a position of the prosthetic valve 10 upon the support surface 310. The ring body 306 accordingly may act as a spacer configured to define a position of the prosthetic valve 10 upon the valve support member 304. In other words, an axial width 371 of the ring body 306 can define a spacing of the prosthetic valve 10 from the second (proximal) end 302p of the coupling member 302.

In some examples, the ring body 306 can be oriented in an open configuration with the arms 362, 364 open and then may be positioned on and around the valve support member 304 with the arms 362, 364 closed to secure the ring body 306 around the valve support member 304. In certain examples, the ring body 306 may be positioned upon the neck portion 338.

The prosthetic valve 10 may then be positioned around the valve support member 304 and the support surface 310, and abutting against the first surface 352 of the ring body 306. The prosthetic valve 10 may be positioned upon the support surface 310 with the commissures 22a-c circumferentially aligned with the indicators 360a-c and an end of the prosthetic valve 10 abutting the first surface 352 (FIG. 12).

The use of the ring body 306 may allow the commissures 22a-c of the prosthetic valve 10 to be positioned in a desired circumferential orientation relative to the ring body 306 and thus relative to the coupling member 302 (for example, relative to the projection 324 of the coupling member 302). The projection 324 can then rotationally align the coupling member 302 with the crimping device 200, and thus place the commissures 22a-c of the prosthetic valve 10 in a desired rotational orientation within the crimping device 200.

As a result, the prosthetic valve 10 can be crimped onto the delivery apparatus at a predetermined circumferential orientation relative to the delivery apparatus (for example, relative to a radiopaque marker on the delivery apparatus, as described herein).

### Example Positioning Device

The coupling member 302, the valve support member 304, and the ring body 306 may each be part of an assembly (for example, mounting assembly 300) or system for use in crimping a prosthetic valve having one or more leaflets to a delivery apparatus. In some examples, the assembly or system may include a positioning device 308 configured to couple to a portion (for example, distal end portion) of the delivery apparatus, proximal to the valve mounting portion. FIG. 11, for example, illustrates an example positioning device 308 positioned proximal of the valve mounting portion 124. The positioning device 308 includes a body 380 including a first portion 382 and a second portion 384 joined at a hinge 386. The body 380 can include a central channel 388 that an intermediate shaft 106 (or another shaft portion, such as the outer shaft 104) of the delivery apparatus may be positioned in, with the second portion 384 rotating about the hinge 386 to close the central channel 388 and retain the delivery apparatus (for example, the intermediate shaft 106) within the central channel 388.

The body 380 may further include a flange portion 390 including one or more mating surfaces (for example, interfaces) in the forms of flanges 392 that are configured to engage the mating structures 296 of the proximal face 292 of the crimping device 200.

The positioning device 308 may be utilized to couple to the distal end portion 109 of the delivery apparatus and suspend the distal end portion 109 of the delivery apparatus in position within the channel 290 of the crimping device 200 (FIGS. 13-14). The positioning device 308 accordingly may hold the delivery apparatus spaced from the pressing surfaces 284 of the crimping device 200, as shown in FIG. 13 for example.

Further, the positioning device 308 may be positioned axially along the delivery apparatus such that the valve mounting portion 124 is held within a defined axial position within the channel 290 of the crimping device 200. In some examples, such a feature may further allow the distal shoulder 126 of the delivery apparatus to be positioned outside of the channel 290 of the crimping device 200 and distal of the channel 290 such that the distal shoulder 126 is not pressed by the pressing surfaces 284 during crimping. The delivery apparatus may further be held in a defined axial position relative to the prosthetic valve 10 positioned upon the valve support member 304 (FIG. 13).

### Example Method of Crimping a Prosthetic Valve

An exemplary method of operation of the systems disclosed herein may include the following steps. Steps may be modified, excluded, or substituted across examples as desired.

Initially, the ring body 306 can be positioned upon the valve support member 304 in a configuration shown in FIG. 4 for example. Specifically, the valve support member 304 can be inserted into the lumen 318 of the coupling member 302 by aligning the recess 340 on the support portion 334 with the cut-out 320 on the coupling member 302. The ring body 306 can be rotationally oriented upon the valve support member 304 in a defined position, for example, via the coupling of the coupler 322 with the cut-out 320. As such, the prosthetic valve 10 may be positioned upon the support surface 310 with the commissures 22a-c of the prosthetic valve 10 circumferentially aligned with the indicators 360a-c (as shown in FIG. 12). The prosthetic valve 10 may abut against the ring body 306.

With the prosthetic valve 10 positioned upon the support surface 310, in the desired rotational alignment, the ring body 306 can then be removed from the valve support member 304, prior to crimping the prosthetic valve 10 to the delivery apparatus 100. For example, the levers 376, 378 may be pressed to rotate the arms 362, 364 about the pivot 370 and open the ring body 306.

With the ring body 306 removed, the valve support member 304 and the coupling member 302 may be inserted into the crimping device 200 with the prosthetic valve 10 positioned around the valve support member 304. FIG. 13, for example, illustrates the prosthetic valve 10 positioned on and around the valve support member 304, which is inserted into the channel 290 of the crimping device 200. The distal opening 206 of the crimping device 200 may be configured for the valve support member 304 and the coupling member 302 to be inserted into the channel 290. The channel 290 of the crimping device 200 may be configured to receive the prosthetic valve 10, the valve support member 304, the coupling member 302, and the distal end portion 109 of the delivery apparatus.

Upon insertion of the valve support member 304 into the channel 290 of the crimping device 200, the projection 324 can be aligned with (for example, received within) the cut-out portion 208 of the crimping device 200. As such, the rotational orientation of the valve support member 304 within the channel 290 of the crimping device 200, and accordingly the rotational orientation of the prosthetic valve 10 within the channel 290 of the crimping device 200, can be set in the desired position.

With the valve support member 304 and the prosthetic valve 10 inserted into the channel 290 of the crimping device 200, the positioning device 308 can be coupled to the distal end portion 109 of the delivery apparatus and then inserted into the proximal opening 294 of the crimping device 200 (FIG. 13). The flanges 392 of the positioning device 308 can mate with the corresponding mating structures 296.

FIG. 13 illustrates a cross sectional view of the pressing surfaces 284 in position around the channel 290 of the crimping device 200, as well as the coupling member 302 and the valve support member 304 inserted into the channel 290 with the prosthetic valve 10 positioned around the valve support member 304.

As shown in FIG. 13, the valve support member 304 extends axially within the channel 290, toward the proximal opening 294 of the crimping device 200. The support surface 310 may be surrounded by the pressing surfaces 284. The coupling member 302 can be arranged outside of and distal of the pressing surfaces 284, and may be retained within the distal opening 206 of the crimping device 200. The projection 324 may extend proximally into the cut-out portion 208 of the crimping device 200.

In FIG. 13, the valve mounting portion 124 of the delivery apparatus is positioned within the channel 290 of the crimping device 200. The prosthetic valve 10 is positioned within the channel 290 and around the valve mounting portion 124 of the delivery apparatus. The valve support member 304 is positioned within the channel 290 and between the prosthetic valve 10 and the delivery apparatus 100. The valve support member 304 supports the leaflets of the prosthetic valve 10 in an open position. The distal end portion 109 of the delivery apparatus extends distally within the interior channel 290 of the crimping device 200 and distally within the central channel 344 of the valve support member 304.

When inserted into the crimping device 200, the positioning device 308 may be coupled to the distal end portion 109 of the delivery apparatus, proximal to the valve mounting portion 124, and may be engaged with the mating structures 296 of the proximal face 292. The positioning device 308 may be coupled to the distal end portion 109 of the delivery apparatus at a location such that the valve mounting portion 124 is positioned at a desired location within the channel 290 and relative to the prosthetic valve 10. For example, as shown in FIG. 13, the prosthetic valve 10 may surround the valve mounting portion 124.

As described above, the rotational alignment of the prosthetic valve 10 relative to the distal end portion 109 of the delivery apparatus may be in a desired, predetermined orientation and/or position due to the prior use of the ring body 306.

With the distal end portion 109 of the delivery apparatus, valve support member 304, and prosthetic valve 10 in a desired position within the channel 290, the actuator of the crimping device 200 may be actuated to compress the prosthetic valve 10. For example, the handle 288 may be rotated to rotate the rotatable body 298 and move the pressing surfaces 284 radially inward against the prosthetic valve 922.

FIG. 14, for example, illustrates the pressing surfaces 284 having been moved radially inward to apply a compressive force to the prosthetic valve 10. The prosthetic valve 10 is crimped to the delivery apparatus, around the valve mounting portion 124, utilizing the pressing surfaces 284 of the crimping device 200. As shown in FIG. 14, in its radially compressed state, the prosthetic valve 10 has increased in length, in the axial direction.

Crimping the prosthetic valve 10 to the delivery apparatus may include applying a force to the support surface 310 of the valve support member 304 with the pressing surfaces 284, thereby causing the valve support member 304 to slide axially within the channel 290, away from the prosthetic valve 10 (FIG. 14).

For example, as described above, the tapered shape of the valve support member 304 and the support surface 310 can cause the valve support member 304 to slide distally away from the channel 290 and away from the pressing surfaces 284, as the pressing surfaces 284 move radially inward. Thus, crimping the prosthetic valve 10 can cause the valve support member 304 (and the support surface 310) to be ejected out of the prosthetic valve 10.

As described above, an axial locking mechanism of the mounting assembly 300 can prevent the coupling member 302 from moving axially relative to the crimping device 200 when the valve support member 304 moves axially relative to the coupling member 302. Thus, the coupling member 302 can remain coupled to the crimping device 200 as the valve support member 304 slide in a direction axially away from the channel 290 upon the crimping device 200 crimping the prosthetic valve 10. In other words, the valve support member 304 can move distally into the lumen 318 of the coupling member 302, which remain fixed relative to the crimping device 200 during crimping (thus the coupling member 302 and the valve support member 304 will not fall off the crimping device 200). Further, as described above, the direction control mechanism of the mounting assembly 300 can ensure unidirectional movement of the valve support member 304 relative to the coupling member. For example, the valve support member 304 can move from the extended state (see, for example, FIGS. 8 and 13) to the retracted state (see, for example, FIGS. 9 and 14), but not vice versa.

Following the prosthetic valve 10 being crimped to the delivery apparatus, the mounting assembly 300 can be removed. For example, the coupling member 302 (and the valve support member 304 thereof in the retracted state) can be removed by unlocking the coupling member 302 from the crimping device 200, for example, by pushing or pulling the coupling member 302 away from the crimping device 200 with a sufficient force to overcome the resistance of the axial locking mechanism, as described above. Additionally, the positioning device 308 can be disengaged from the mating structures 296 and moved outward from the proximal opening 294, thereby moving the delivery apparatus outward and away from the crimping device 200. The positioning device 308 can then be removed from the distal end portion 109 of the delivery apparatus, with the prosthetic valve 10 crimped to the delivery apparatus.

In this way, the use of a mounting assembly 300 including the valve support member 304 and the coupling member 302 may allow the leaflets of the prosthetic valve 10 to remain in an open position during crimping. Such a feature may reduce the possibility of degradation to the prosthetic valve 10 occurring during crimping. Further, the tapered shape of the support surface 310 may allow the valve support member 304 to be slid outward from the crimping device via the radially inward movement of the pressing surfaces 284, such that the valve support member 304 automatically is moved outward and away from the crimped prosthetic valve 10. The valve support member 304 may automatically slide axially outward such that the support surface 310 is not positioned between the prosthetic valve 10 and the pressing surfaces 284 following crimping. In some examples, the system may be configured such that a separate mechanism slides the valve support member 304 distally, such that a tapered shape may not be utilized for the support surface 310. For example, arms or gears or another form of coupler may engage the valve support member 304 to move the valve support member 304 away from the prosthetic valve 10. Furthermore, the coupling member 302 can remain fixedly attached to the crimping device (without falling) while the valve support member 304 moves unidirectionally from the extended state to the retracted state.

Although the mounting assembly 300 described above has a specific linear ratchet mechanism that enables the valve support member 304 to move distally relative to the coupling member 302, it is to be understood that the mounting assembly can have different configurations that still allow the valve support member to move from the extended state to the retracted state when crimping the prosthetic valve. For example, the valve support member can have a plurality of concentrically arranged telescopic shafts. The outermost shaft, which interfaces with the pressing surfaces of the crimping device, can have a similar conical shape as the support portion 334, which is configured to be inserted into the prosthetic valve and support the leaflets of the prosthetic valve in an open position. Each inner shaft can be moved distally relative to an immediately adjacent outer shaft. Thus, by progressively moving the shafts (from the inner most shaft to the outer most shaft) in the distal direction (relative to the coupling member), the valve support member can be moved from an extended state to a retracted state when crimping the prosthetic valve, thereby ejecting the valve support member out of the prosthetic valve and allowing the prosthetic valve to be crimped onto the delivery apparatus.

Alternate examples of mounting assemblies that are configured to crimp a prosthetic valve onto a delivery apparatus at a predetermined position and/or orientation relative to the delivery apparatus are described in International Patent Application WO 2022/046585, which is incorporated by reference herein.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A mounting assembly for crimping a prosthetic valve onto a valve mounting portion of a delivery apparatus, the mounting assembly comprising: a coupling member configured to releasably couple to a crimping device, the coupling member comprising a lumen; and a valve support member configured to be inserted into the prosthetic valve so that leaflets of the prosthetic valve contact and rest upon a support surface of the valve support member, wherein the valve support member is axially movable relative to the coupling member through the lumen of the coupling member.

Example 2. The mounting assembly of any example herein, particularly example 1, wherein the valve support member comprises a connect portion, a support portion, and a neck portion located between the connect portion and the support portion, wherein the support portion defines the support surface.

Example 3. The mounting assembly of any example herein, particularly example 2, wherein the support portion has a conical shape with a diameter decreasing from a distal end of the support portion to a proximal end of the support portion.

Example 4. The mounting assembly of any example herein, particularly example 3, wherein the neck portion has a cylindrical shape with a diameter smaller than the distal end of the support portion and a proximal end of the connect portion.

Example 5. The mounting assembly of any example herein, particularly any one of examples 2-4, further comprising a direction control mechanism configured to allow the valve support member to move distally relative to the coupling member and prevent the valve support member from moving proximally relative to the coupling member.

Example 6. The mounting assembly of any example herein, particularly example 5, wherein the direction control mechanism comprises at least one ridge extending radially outwardly from the connect portion of the valve support member and a plurality of teeth located on an inner surface of the coupling member, wherein the at least one ridge is configured to matingly engage with the plurality of teeth.

Example 7. The mounting assembly of any example herein, particularly example 6, wherein the connect portion of the valve support member comprises a plurality of axially extending arms arranged in a circumferential direction and separated by a plurality of axially extending gaps, wherein each axially extending arm comprises a corresponding ridge extending radially outwardly from the arm.

Example 8. The mounting assembly of any example herein, particularly any one of examples 6-7, wherein the connect portion of the valve support member defines a first diameter when the at least one ridge is engaged with one of the teeth, wherein the connect portion of the valve support member is configured to deflect radially inwardly so as to define a second diameter when the at least one ridge moves across two adjacent teeth, wherein the second diameter is smaller than the first diameter.

Example 9. The mounting assembly of any example herein, particularly any one of examples 6-8, further comprising a bias member inserted into the connect portion of the valve support member, wherein the bias member is configured to press the connect portion radially outwardly against the inner surface of the coupling member so as to prevent the at least one ridge from rattling when the at least one ridge matingly engages with the plurality of teeth.

Example 10. The mounting assembly of any example herein, particularly example 9, wherein the bias member comprises a coiled spring.

Example 11. The mounting assembly of any example herein, particularly any one of examples 2-10, wherein the valve support member comprises a central channel configured for the delivery apparatus to extend therethrough.

Example 12. The mounting assembly of any example herein, particularly any one of examples 2-11, further comprising a rotation locking mechanism configured to prevent the valve support member from rotating relative to the coupling member.

Example 13. The mounting assembly of any example herein, particularly example 12, wherein the rotation locking mechanism comprises a key extending axially along an inner surface of the coupling member and a channel formed on the connect portion of the valve support member, wherein the channel is configured to receive the key.

Example 14. The mounting assembly of any example herein, particularly any one of examples 4-13, further comprising a ring body configured to couple to and extend around the neck portion, wherein the ring body comprises a first arm and a second arm, wherein a first end portion of the first arm is pivotably connected to a first end portion of the second arm, wherein a second end portion of the first arm is releasably connected to a second end portion of the second arm.

Example 15. The mounting assembly of any example herein, particularly example 14, wherein the coupling member comprises a cut-out, the support portion comprises a recess, and the ring body comprises an axially extending coupler, wherein the cut-out is configured to rotationally align with the recess so that the axially extending coupler is received within the cut-out and the recess.

Example 16. The mounting assembly of any example herein, particularly any one of examples 1-15, wherein the coupling member comprises an alignment member configured to rotationally align the coupling member with the crimping device.

Example 17. The mounting assembly of any example herein, particularly example 16, wherein the alignment member comprises an axially extending lever configured to be inserted into a cut-out portion of the crimping device.

Example 18. The mounting assembly of any example herein, particularly example 17, further comprising an axial locking mechanism configured to prevent the coupling member from moving axially relative to the crimping device when the valve support member moves axially relative to the coupling member.

Example 19. The mounting assembly of any example herein, particularly example 18, wherein the axial locking mechanism comprises a protrusion extending radially outwardly from the axially extending lever and a recess connected to the cut-out portion, wherein the protrusion is configured to snap into the recess.

Example 20. The mounting assembly of any example herein, particularly any one of examples 1-10, further comprising the crimping device.

Example 21. A mounting assembly for crimping a prosthetic valve onto a valve mounting portion of a delivery apparatus, the mounting assembly comprising: a coupling member configured to releasably couple to a crimping device; and a valve support member comprising a connect portion inserted into the coupling member and a support surface, wherein the prosthetic valve can slide over the support surface so that leaflets of the prosthetic valve contact and rest upon the support surface, wherein the valve support member is axially movable between an extended state and a retracted state, wherein the support surface extends out of the coupling member when the valve support member is in the extended state, wherein the support surface is at least partially covered by the coupling member when the valve support member is in the retracted state.

Example 22. The mounting assembly of any example herein, particularly example 21, wherein a distal portion of the support surface has a larger diameter than a proximal portion of the support surface.

Example 23. The mounting assembly of any example herein, particularly any one of examples 21-22, wherein the valve support member is configured to move unidirectionally from the extended state to the retracted state.

Example 24. The mounting assembly of any example herein, particularly any one of examples 21-23, wherein the valve support member is not rotatable relative to the coupling member.

Example 25. A mounting assembly for crimping a prosthetic valve onto a valve mounting portion of a delivery apparatus, the mounting assembly comprising: a coupling member configured to releasably couple to a crimping device; and a valve support member comprising a connect portion inserted into the coupling member and a support surface, wherein the valve support member is movable between an extended state and a retracted state, wherein when the valve support member is in the extended state, the prosthetic valve in a radially expanded configuration can slide over the support surface, wherein when the valve support member is in the retracted state, the support surface is moved away from the prosthetic valve so that the prosthetic valve can be crimped to a radially compressed configuration by the crimping device.

Example 26. A method comprising: placing a prosthetic valve in a radially expanded configuration over a support surface of a valve support member, wherein a connect portion of the valve support member is inserted into a coupling member; inserting the prosthetic vale placed over the support surface into a channel of a crimping device, the channel being surrounded by a plurality of pressing surfaces of the crimping device; attaching the coupling member to the crimping device; and radially compressing the prosthetic valve placed within the channel by actuating the crimping device to move the pressing surfaces radially inwardly, wherein the radially compressing causes the support surface to be ejected out of the prosthetic valve while the coupling member remains fixed relative to the crimping device.

Example 27. The method of any example herein, particularly example 26, further comprising rotationally aligning the prosthetic valve relative to the valve support member.

Example 28. The method of any example herein, particularly any one of examples 26-27, further comprising rotationally aligning the coupling member to the crimping device.

Example 29. The method of any example herein, particularly any one of examples 26-28, further comprising inserting a delivery apparatus through a lumen of the valve support member so that the prosthetic valve placed over the support surface is aligned with a valve mounting portion of the delivery apparatus.

Example 30. The method of any example herein, particularly example 29, further comprising attaching a positioning device to a predefined position of the delivery apparatus and coupling the positioning device to the crimping device, wherein the predefined position is proximal to the valve mounting portion.

Example 31. The method of any example herein, particularly any one of examples 26-30, can be used in implanting the prosthetic valve in a human patient or a non-living simulation object.

Example 32. A method comprising sterilizing the mounting assembly of any example herein, particularly any one of examples 1-25.

The features described herein with regard to any example can be combined with other features described in any one or more of the other examples, unless otherwise stated. For example, any one or more of the features of one delivery apparatus can be combined with any one or more features of another delivery apparatus.

It is to be understood that the treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A mounting assembly for crimping a prosthetic valve onto a valve mounting portion of a delivery apparatus, the mounting assembly comprising:
a coupling member configured to releasably couple to a crimping device, the coupling member comprising a lumen; and
a valve support member configured to be inserted into the prosthetic valve so that leaflets of the prosthetic valve contact and rest upon a support surface of the valve support member,
wherein the valve support member is axially movable relative to the coupling member through the lumen of the coupling member.

2. The mounting assembly of claim 1, wherein the valve support member comprises a connect portion, a support portion, and a neck portion located between the connect portion and the support portion, wherein the support portion defines the support surface.

3. The mounting assembly of claim 2, wherein the support portion has a conical shape with a diameter decreasing from a distal end of the support portion to a proximal end of the support portion.

4. The mounting assembly of claim 3, wherein the neck portion has a cylindrical shape with a diameter smaller than the distal end of the support portion and a proximal end of the connect portion.

5. The mounting assembly of claim 2, further comprising a direction control mechanism configured to allow the valve support member to move distally relative to the coupling member and prevent the valve support member from moving proximally relative to the coupling member.

6. The mounting assembly of claim 5, wherein the direction control mechanism comprises at least one ridge extending radially outwardly from the connect portion of the valve support member and a plurality of teeth located on an inner surface of the coupling member, wherein the at least one ridge is configured to matingly engage with the plurality of teeth.

7. The mounting assembly of claim 6, wherein the connect portion of the valve support member comprises a plurality of axially extending arms arranged in a circumferential direction and separated by a plurality of axially extending gaps, wherein each axially extending arm comprises a corresponding ridge extending radially outwardly from the arm.

8. The mounting assembly of claim 6, wherein the connect portion of the valve support member defines a first diameter when the at least one ridge is engaged with one of the teeth, wherein the connect portion of the valve support member is configured to deflect radially inwardly so as to define a second diameter when the at least one ridge moves across two adjacent teeth, wherein the second diameter is smaller than the first diameter.

9. The mounting assembly of claim 6, further comprising a bias member inserted into the connect portion of the valve support member, wherein the bias member is configured to press the connect portion radially outwardly against the inner surface of the coupling member so as to prevent the at least one ridge from rattling when the at least one ridge matingly engages with the plurality of teeth.

10. The mounting assembly of claim 2, further comprising a rotation locking mechanism configured to prevent the valve support member from rotating relative to the coupling member.

11. The mounting assembly of claim 10, wherein the rotation locking mechanism comprises a key extending axially along an inner surface of the coupling member and a channel formed on the connect portion of the valve support member, wherein the channel is configured to receive the key.

12. The mounting assembly of claim 1, wherein the coupling member comprises an alignment member configured to rotationally align the coupling member with the crimping device.

13. The mounting assembly of claim 12, wherein the alignment member comprises an axially extending lever configured to be inserted into a cut-out portion of the crimping device.

14. The mounting assembly of claim 13, further comprising an axial locking mechanism configured to prevent the coupling member from moving axially relative to the crimping device when the valve support member moves axially relative to the coupling member.

15. The mounting assembly of claim 14, wherein the axial locking mechanism comprises a protrusion extending radially outwardly from the axially extending lever and a recess connected to the cut-out portion, wherein the protrusion is configured to snap into the recess.

16. A mounting assembly for crimping a prosthetic valve onto a valve mounting portion of a delivery apparatus, the mounting assembly comprising:
a coupling member configured to releasably couple to a crimping device; and
a valve support member comprising a connect portion inserted into the coupling member and a support surface,
wherein the prosthetic valve can slide over the support surface so that leaflets of the prosthetic valve contact and rest upon the support surface,
wherein the valve support member is axially movable between an extended state and a retracted state,
wherein the support surface extends out of the coupling member when the valve support member is in the extended state,
wherein the support surface is at least partially covered by the coupling member when the valve support member is in the retracted state.

17. The mounting assembly of claim 16, wherein a distal portion of the support surface has a larger diameter than a proximal portion of the support surface.

18. The mounting assembly of claim 16, wherein the valve support member is configured to move unidirectionally from the extended state to the retracted state.

19. The mounting assembly of claim 16, wherein the valve support member is not rotatable relative to the coupling member.

20. A method comprising:
placing a prosthetic valve in a radially expanded configuration over a support surface of a valve support member, wherein a connect portion of the valve support member is inserted into a coupling member;
inserting the prosthetic vale placed over the support surface into a channel of a crimping device, the channel being surrounded by a plurality of pressing surfaces of the crimping device;
attaching the coupling member to the crimping device; and
radially compressing the prosthetic valve placed within the channel by actuating the crimping device to move the pressing surfaces radially inwardly,
wherein the radially compressing causes the support surface to be ejected out of the prosthetic valve while the coupling member remains fixed relative to the crimping device.
